(19) United States
(12) Patent Application Publication
(10) Pub. No.: US 2024/0366192 A1
(43) Pub. Date: Nov. 7, 2024

(54) COMPUTER IMPLEMENTED METHOD, COMPUTER SYSTEM AND COMPUTER PROGRAM PRODUCT FOR DETERMINING A MENOPAUSAL STATE

(71) Applicant: identifyHer Ltd., Dublin (IE)

(72) Inventors: Donal O’GORMAN, Dublin (IE); Alish KERR, Dublin (IE); Heidi DAVIS, Dublin (IE)

(21) Appl. No.: 18/654,072

(22) Filed: May 3, 2024

(30) Foreign Application Priority Data
May 4, 2023 (EP) 23171642.4

(51) Int. Cl. A61B 10/00; G06N 3/044; G06N 3/0464

(52) U.S. Cl. CPC A61B 10/0012; G06N 3/044; G06N 3/0464; A61B 2010/0016; A61B 2010/0019

(57) ABSTRACT

The invention relates to a computer implemented method, a computer system (1), and a computer program product for determining a menopausal state, and particularly relates to such a method, system (1) and computer program product that employs sensors measuring data and processing the measured data by means of a self-learning classification model (43). Disclosed is a computer implemented method for providing a model for determining a menopausal state, comprising the steps: determining a set of measurable body conditions from a group of body conditions of a human body, the body conditions being indicative of a menopausal state of a human body (5), determining a set of training objects, the training objects being humans being capable of adopting a menopausal state and having a known state concerning their menopausal state as menopausal state information, measuring the measurable body conditions of the set of measurable body conditions of each of the training objects for a predetermined amount of time to provide measured body condition information for each of the training objects, preprocessing the measured body condition information to provide preprocessed body condition information, providing a computer implemented classification model (43) adapted to classify a menopausal state, inputting the preprocessed body condition information of a training object as training input information to the classification model (43), inputting the menopausal state information of the test object as training classification information to the classification model (43), adapting the classification model (43) according to the training input information and training classification information.

This application claims priority to European Patent Application No. 23171642.4 filed May 4, 2024 entitled “COMPUTER IMPLEMENTED METHOD, COMPUTER SYSTEM AND COMPUTER PROGRAM PRODUCT FOR DETERMINING A MENOPAUSAL STATE”. The entire contents of this application are herein incorporated by reference.

The invention relates to a computer implemented method, a computer system, and a computer program product for determining menopausal state, and in particular, relates to such a method, system and computer program product that employs sensors measuring data and processing the measured data by means of a self-learning model.

In the prior art, the basic principles of analyzing data with self-learning models or similar analysis methods in a medical context is known. A variety of different sensors are used to capture biophysical data signals which are then analyzed using intelligent computing methods like artificial neural networks and the like.

In another example, so-called symptom checkers analyze static patient record data as well as provided symptoms from interactive chats with patients deriving probabilities of relevant diseases.

In another example, glucose measurement for diabetes patients also uses sensor technologies to continuously measure values of blood parameters that can be used for deriving predictions and recommendations for treatment and medication.

In terms of menopause symptom diagnosis and treatment, mainly symptom treatment is known in prior art such as cooling devices, blankets, or special clothing. In another example, heart rate sequence data analysis is used for determining a hot flash using a sensor device.

EP3591659A1 describes a system for managing hot flashes and menopause symptoms using a sensor circuitry in combination with a logic circuitry having a predictive model that indicates a probability of a person having a hot flash.

Some menopausal symptoms, however, are in general unspecific or not unique to menopause. They may occur in the context of several pathological or non-pathological conditions. Although certain principles of detecting different symptoms that inter alia occur with menopause are known in prior art, one of the main challenges remains the clear identification of menopausal symptoms as determining a menopausal state.

Menopausal symptoms indicating a menopausal state occur with large time spans and provide the challenge of large variations of possible symptom characteristics. More than 40 different possible symptoms are known to be related to menopause and occur in different compositions, frequency and severity.

Menopausal symptoms, in particular perimenopausal symptoms may include vasomotor symptoms like hot flashes and night sweat, insomnia, decreased cognitive performance, depressive states, migraine, weight gain, anxiety, joint aches, palpitation, vaginal dryness, urinary symptoms and low libido. Postmenopause may result in an increase of FSH level, change in blood lipid profile, increased glucose intolerance, bone demineralization, increased blood pressure, osteoporosis and osteoarthritis.

As menopausal symptoms, in particular perimenopausal symptoms overlap with stressful states or anxiety states and in addition strongly vary in terms of timely occurrence (larger period of time) and intensity, as well as in their characteristics as a combination of a variety of different particular single body symptoms. Automated prediction and detection accuracy remains a technical challenge. In particular, an accurate differentiation of hot flushes to other comparable events and activities is not yet sufficiently solved. For example, an increased mean heart rate as well as an increased mean temperature, both per se and combined may be indicative of a menopausal state. However, if they occur together, they may be also caused by other factors like exercising.

At the same time, medical staff and physicians expect a medical-grade accuracy and not just a certain probability of a menopausal symptom.

Document Tsiartas, Baker, Smith, Zambotti, “A novel Hot-Flash classification algorithm via multi-sensor features integration”, EMBC, Oct. 31-Nov. 4 2021, PMID 34891695 discloses a hot flash classification algorithm based on multi-sensor features integration using commercial wearable wrist worn sensors, aggregating photoplethysmographic (PPG), motion, temperature and skin conductance data. Via a decision tree, hot flashes are recorded and assessed. Sensor information is computed using ±15 second windows. Skin conductance features are compared to ±2 minutes around a hot flash or 250 seconds prior to the hot flash and the current 30 seconds window. Temperature features are compared between prior and following 500 seconds of a hot flash. However, the teaching requires a large amount of computational resources and provides insufficiently accurate results.

It can be seen as an object of the invention to provide a computer implemented method, a computer system, and a computer program product that solve the problems in the prior art, and in particular to provide a computer implemented method, a computer system and a computer program product for efficiently, securely and reliably determining a menopausal state.

The object is solved by the subject-matter of the independent claims. Advantageous further developments are subject-matter of the dependent claims.

Disclosed is a computer implemented method for providing a model for determining a menopausal state, comprising the steps: a) determining a set of measurable body conditions from a group of body conditions of a human body, the body conditions being indicative of a menopausal state of a human body, b) determining a set of training objects, the training objects being humans being capable of adopting a menopausal state and having a known state concerning their menopausal state as menopausal state information, c) measuring the measurable body conditions of the set of measurable body conditions of each of the training objects for a predetermined amount of time to provide measured body condition information for each of the training objects, and detecting occurrence of a symptom of a menopausal state, d) preprocessing the measured body condition information to provide preprocessed body condition information, e) providing a computer implemented classification model adapted to classify a menopausal state, f) inputting the preprocessed body condition information of a training object as training input information to the classification model, g) inputting the detected occurrence of the symptom of a menopausal state as training classification information to the classification model, h) adapting the classification model according to the training input information and training classification information.

Advantageously, in step c), the measured body condition information is a time series of measured body conditions, respectively.

Advantageously, in step d) the computer implemented classification model is a decision tree model or a random forest model or an artificial neural network model, in particular a recurrent neural network model or a convolutional neural network model, further in particular an echo state network model or a liquid state machine model.

Advantageously, in step a), the set of body condition information contains a subset of the set: electrodermal activity, heart rate, in particular photoplethysmographic information and electrocardiogram information, body temperature, body movement activity, in particular relocation or acceleration, body environment information, in particular ambient temperature, ambient humidity or ambient pressure, Bioimpedance, EMG, EEG, Ultrasound, NIRS, Microbiota (vaginal or gut), pH, Voice patterns, Breathing pattern and frequency, Interstitial fluid deviations.

Advantageously, step c) further comprises the step: ca) measuring electrodermal activity and providing measured electrodermal activity information, and step d) further comprises the steps: da) transforming the measured electrodermal activity information into SCL information and SCR information, and db) providing the SCL information or the SCR Information as measured body condition information, and step f) comprises the step: fa) inputting the SCL information or the SCR information as measured body condition information to the classification model.

Advantageously, step h) further comprises the step: ha) providing the SCL information with the highest weight of the set of measurable body conditions for classification.

Advantageously, step d) further comprises at least one of the steps: dc) applying a low pass filter, in particular a Butterworth filter, in particular a Butterworth filter with a cut off frequency of 0.5 Hz, to the measured body condition information, dd) adapting a sampling rate of the measured body condition information to 1 Hz for the measurable body conditions of the set of measurable body conditions, de) smoothen the measured body condition information by applying a sliding left windows of 60 seconds, in particular by determining a simple moving average by forming the unweighted mean of the previous 60 measured samples, df) providing the measured body condition information with classification information such that the measured body condition information that occur within a time period of 180 seconds preceding and of 180 seconds succeeding a point in time of the detection of a symptom of a menopausal state are labeled as belonging to the symptom of the menopausal state, and that the measured body condition information that occur outside that time period are labeled as not belonging to a symptom of a menopausal state.

Advantageously, step d) further comprises step: dg) applying an additional filter to the measured body condition information based on measured body condition information.

Advantageously, in step dg) measured EDA information or measured T information is filtered if it occurs together with increased measured ambient temperature information or increased measured ACC information.

Advantageously, the classification model is located at a remote site, in particular, as a centralized cloud service.

Disclosed is a computer implemented method for determining a menopausal state, comprising the steps: i) providing a classification model according to any of claims 1 to 10, j) determining a diagnose object, the diagnose object being a human being capable of adopting a menopausal state and having an unknown state concerning their menopausal state as menopausal state information, k) arranging at least one sensor at the body of the diagnose object at a sensor body location, l) measuring, by means of the at least one sensor, a set of measurable body conditions from a group of body conditions of a human body, the body conditions being indicative of a menopausal state of a human body and being a subset of the set of measurable body conditions according to step a), as measured diagnose body condition information, m) preprocessing the measured diagnose body condition information to provide preprocessed diagnose body condition information, n) inputting the preprocessed diagnose body condition information of the diagnose object as diagnose input information to the classification model, o) classifying a menopausal state of the diagnose object as menopausal state information, p) using the classified menopausal state information for providing a diagnose on the menopausal state of the diagnose object.

Advantageously, in step l), the measured diagnose body condition information is a time series of measured body conditions, respectively.

Advantageously, in step i) the computer implemented classification model is a decision tree model or a random forest model or an artificial neural network model, in particular a recurrent neural network model or a convolutional neural network model, further in particular an echo state network model or a liquid state machine model.

Advantageously, in step l), the set of body condition information contains a subset of the set: electrodermal activity, heart rate, in particular photoplethysmographic and electrocardiogram (ECG) information, body temperature, body movement activity, in particular relocation or acceleration, body environment information, in particular ambient temperature, ambient humidity or ambient pressure, Bioimpedance, EMG, EEG, Ultrasound, NIRS, Microbiota (vaginal or gut), pH, Voice patterns, Breathing pattern and frequency, Interstitial fluid deviations.

Advantageously, step l) further comprises the step: la) measuring electrodermal activity and providing measured diagnose electrodermal activity information, and step m) further comprises the steps: ma) transforming the measured diagnose electrodermal activity information into SCL information and SCR information, and mb) providing the SCL information or the SCR Information as measured diagnose body condition information, and step n) comprises the step: na) inputting the SCL information or the SCR information as measured diagnose body condition information to the classification model.

Advantageously, step o) further comprises the step: oa) considering the SCL information with the highest weight of the set of measurable body conditions for classification.

Advantageously, step m) further comprises at least one of the steps: mc) applying a low pass filter, in particular a Butterworth filter, in particular a Butterworth filter with a cut-off frequency of 0.5 Hz, to the measured diagnose body condition information, md) adapting a sampling rate of the measured diagnose body condition information to 1 Hz for the measurable body conditions of the set of measurable body conditions, me) smoothen the measured diagnose body condition information by applying a sliding left windows of 60 seconds, in particular by determining a simple moving average by forming the unweighted mean of the previous 60 measured samples, or mf) applying an additional filter to the measured diagnose body condition information based on measured diagnose body condition information.

Advantageously, the method further comprises the step: dg) providing a sensor unit at a sensor location, wherein the sensor location is arranged at a human body, in particular at a torso, in particular at the lower thorax, in particular below the sternum, either centrally or at the frontal left side of the thorax of the diagnose object.

Disclosed is a computer system for determining a menopausal state, comprising: a sensor unit, a transmission unit, and an evaluation unit, wherein
the sensor unit is configured to measure a set of measurable body conditions from a group of body conditions of a human body, the body conditions being indicative of a menopausal state of a human body and being a subset of the set of measurable body conditions and transmit measured body conditions as measured body condition information to the transmission unit, the transmission unit is configured to receive the body condition information from the sensor unit and to transmit the body condition information to the evaluation unit, and the sensor unit is configured to perform the step l) and the evaluation unit is configured to perform the steps n) and o) when the system for determining a menopausal state is applied in the computer implemented method for determining a menopausal state.

Advantageously, the sensor unit, the transmission unit or the evaluation unit is configured to perform step m).

Advantageously, the system is configured to perform any of the previous computer implemented methods for determining a menopausal state.

Advantageously, the sensor unit further comprises a sensing portion at a proximal side of the sensor unit, the sensing portion comprising a convex shape, and an urging portion that is configured to urge the sensing portion towards a dermal surface at a sensor location, such that the sensing portion indents the dermal surface.

Advantageously, the/a sensor location is arranged at a human body, in particular at a torso, in particular at the lower thorax, in particular below the sternum, either centrally or at the frontal left side of the thorax.

Disclosed is a computer implemented method for establishing a weighted set of measurable body conditions, comprising the steps: p) providing a classification model according to any of claims 1 to 10, q) determining a set of control objects, the control objects being humans being capable of adopting a menopausal state and having a known state concerning their menopausal state as menopausal state information, r) arranging at least one sensor at the body of the control object at a sensor body location, s) measuring, by means of the at least one sensor, a set of measurable body conditions from a group of body conditions of a human body, the body conditions being indicative of a menopausal state of a human body and being a subset of the set of measurable body conditions according to step a), as measured control body condition information, t) preprocessing the measured control body condition information to provide preprocessed control body condition information, u) inputting the preprocessed control body condition information of the control object as control input information to the classification model, v) classifying a menopausal state of the control object as classified menopausal state information, w) comparing the classified menopausal state information with the menopausal state information of the control object, x) determining a significance of a member of the set of measurable body conditions for classification of the menopausal state for all members of the set of measurable body conditions, y) providing a weight of the measured body condition information corresponding to the significance of the measurable body condition and adapting the classification model according to the weights of the measured body conditions.

Advantageously, the method further comprises the step z) reducing the set of measurable body conditions by removing an at least one least significant member.

Advantageously, step s) further comprises the step: sa) measuring electrodermal activity and providing measured electrodermal activity information, and step t) further comprises the steps: ta) transforming the measured electrodermal activity information into SCL information and SCR information, and tb) providing the SCL information or the SCR Information as measured body condition information, and step u) comprises the step: ua) inputting the SCL information or the SCR information as measured body condition information to the classification model.

Advantageously, step y) further comprises the step: ya) providing the SCL information with the highest weight of the set of measurable body conditions for classification.

Disclosed is a computer readable computer program product storing computer executable instructions that, when executed by the computer system, makes the computer system performing any one of the above methods.

Disclosed is a computer implemented system for understanding menopausal progression that is configured to use a sensor circuitry in combination with a logic circuitry, the logic circuitry implementing a predictive model that indicates the time to menopause onset.

Disclosed is a computer implemented system that is configured to use continuous biometric measurements for perimenopausal, menopausal and post-menopausal diagnose objects to derive predictions and recommendations for treatment and medication of menopausal symptoms.

### BRIEF DESCRIPTION OF THE DRAWINGS

In the following, the invention is explained by means of an embodiment and the figures.

FIG. 1 exhibits a schematic view of an embodiment of a computer implemented system for determining a menopausal state.

FIG. 2 exhibits a detailed view of a sensor unit according to the embodiment.

FIG. 3 exhibits a flow chart of an embodiment of a computer implemented method for providing a model for determining a menopausal state.

FIG. 4 exhibits a flow chart of a preprocessing process of the computer implemented method for providing a model for determining a menopausal state.

FIG. 5 exhibits a flow chart of a training process of the computer implemented method for providing a model for determining a menopausal state.

FIG. 6 exhibits a flow chart of an embodiment of a computer implemented method for determining a menopausal state.

FIG. 7 exhibits a flow chart of a preprocessing process of the computer implemented method for determining a menopausal state.

FIG. 8 exhibits a schematic view of positioning of a sensor.

### DETAILED DESCRIPTION OF THE DRAWINGS

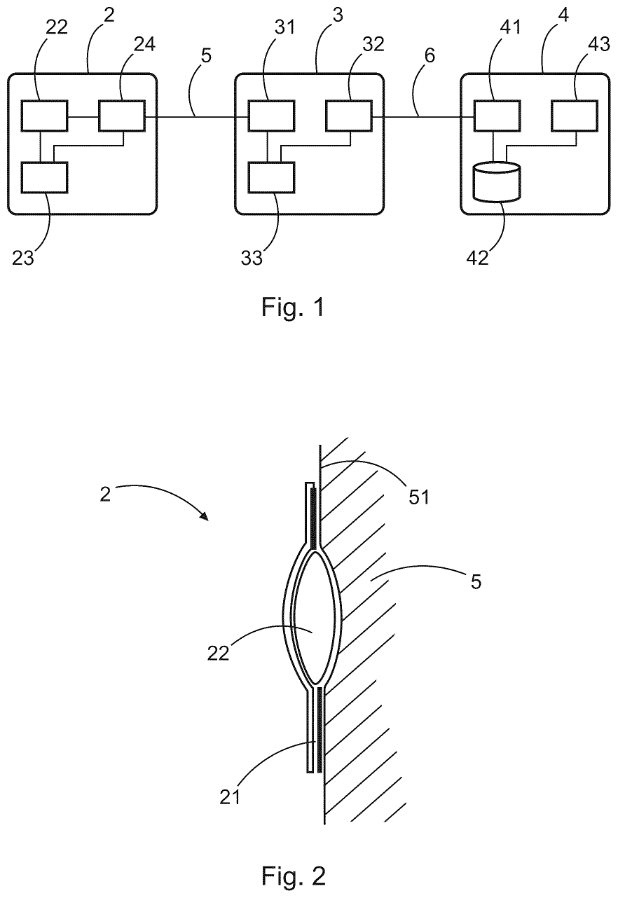

By means of FIG. 1 and FIG. 2, an embodiment of a computer implemented system 1 for determining a menopausal state is described.

The system 1 comprises a sensor unit 2, a transmission unit 3 and an evaluation unit 4.

The sensor unit 2 is a supra dermal wearable sensor that is fixed to a sensor location 52 on a dermal surface 51 of a human body 5 by means of a mechanical fixing method. In the current embodiment, all relevant information may be collected by a supra dermal wearable sensor. In further developments, however, data collected by a intradermal sensor, in particular a needle penetrating the skin for measurements of body fluids may be used. In the embodiment, the fixing method is adhesion by an adhesive layer 21. Alternatively, positive locking, e.g. by means of an elastic belt may be employed.

As shown in FIG. 8, I sensor location 52 in the embodiment is at the torso, preferably at the lower thorax, preferably below the sternum, either centrally or at the frontal left side of the thorax. This sensor location 52 exhibited better sensor signal quality than other sensor location of wearable sensors known from the related art like wrists.

The sensor unit 2 comprises a sensing portion 22 that is capable of detecting photoplethysmographic (PPG) information, electrocardiogram (ECG) information, skin temperature (T) information acceleration/motion (ACC) information and electrodermal activity (EDA) information. PPG information is detected at a sample rate of 50-100 Hz. ECG information is detected at a sample rate of 100-500 Hz. EDA information is detected at a sample rate of 4-30 Hz. T information is detected at a sample rate of 0.2-1 Hz. ACC information is detected at a sample rate of 25-100 Hz. The sensing portion 21 is located at the proximal side of the sensor and comprises a convex projection. Due to the convex shape, the sensing portion 22 is firmly urged towards the skin and thus provides a secure contact area between the skin surface and the sensing portion 22, thus improving the detection of sensor information, in particular improving PPG sensor information.

The sensor unit 2 comprises a sensor communication portion 24 that is configured to establish a wireless short range communication link 6 with the transmission unit 3, e.g. via a transmission protocol according to IEEE 802.15.1 (Bluetooth). Other wireless transmission protocols like protocols according to IEEE 802.11 or proprietary protocols may be used. Raw sensor data is transmitted via the short range communication link 6.

The sensor unit 2 comprises a sensor stored energy source 24, e.g. a rechargeable or non-rechargeable battery, that supplies the sensing portion 21 and the sensor communication portion 24 with electric energy for operation.

The sensor stored energy source 24 is removable from the sensor unit 2 and replaceable to extend the device's battery life when changed. Allowing the user to swap out the battery without removing the sensor unit 2 from the human body 5. The sensor stored energy source 24, when being a rechargeable battery, is recharged in a charging case (not shown) designed specifically to hold the sensor stored energy source 24, the charging case is connected to an electrical outlet through a cable.

The transmission unit 3 is a transceiver that establishes the short-range transmission link 6 with the sensor communication portion 24 via a short-range communication portion 31. The transmission unit 3 preferably is a mobile device, preferably a smart phone. The transmission unit 3 comprises a long-range communication unit 32, e.g. a Wi-Fi module or a cellphone module to establish a wireless long range communication link 7 either directly or indirectly, e.g. via the internet, with the evaluation unit 4, e.g. via a transmission protocol according to IEEE 802.11 or according to GSM, UMTS, LTE or the like. Raw sensor data is transmitted via the long-range communication link 7 to the evaluation unit 4.

The evaluation unit 4 is an electronic data processing device comprising a data storage 42 and implementing a classification model 43. In this embodiment, the classification model 43 is a random forest model. Alternatively, other classification models, in particular decision trees or deep learning methods and artificial neural networks may be employed. The evaluation unit comprises a network interface 42 that is configured to establish the long-range transmission link 7 with the transmission unit 3 via the long-range communication portion 32.

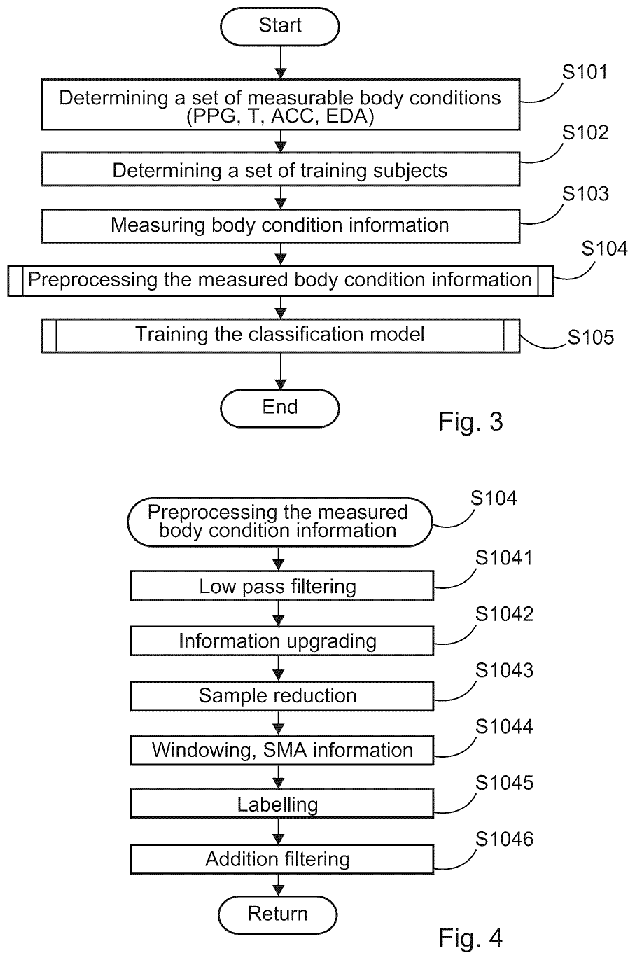

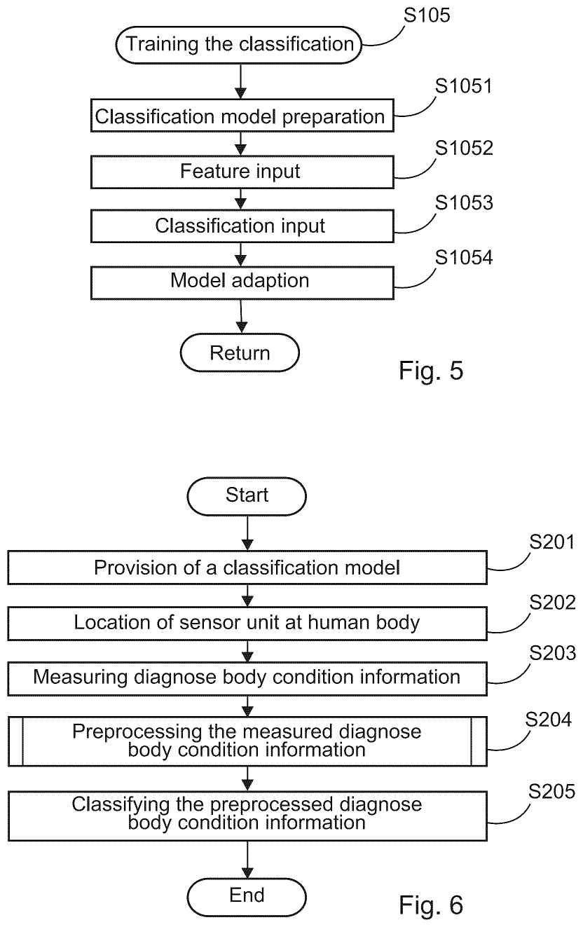

By means of FIG. 3, FIG. 4 and FIG. 5, an embodiment of a computer implemented method for establishing a classification model for determining a menopausal state is described.

FIG. 3 shows a flow chart of an embodiment of a method for establishing a classification model for determining a menopausal state.

In a parameter determining step S101, a set of measurable body conditions from a group of body conditions of a human body are determined as measured parameters that may be indicative of a menopausal state of a human body. In the embodiment, the menopausal state that is intended to be determined is perimenopause. In the embodiment, the set of measurable body conditions corresponds to the available sensor information measured by the sensor unit 2 and output as measured body condition information. In the embodiment, the set of measurable body conditions comprise photoplethysmographic (PPG) properties, electrocardiogram (ECG properties), skin temperature (T) properties, acceleration/motion (ACC) properties and electrodermal activity (EDA) properties.

In a training object determining step S102, a number, that is sufficiently large according to known statistic principles, of human participants is selected as training objects, whereas the menopausal state of the participants is known. I.e. it is known whether a training object is in a perimenopausal state or not.

In a data acquisition step S103, the training objects are provided with sensors, in particular a sensor like the sensor unit 2, to measure and record the set of measurable body conditions, i.e. to measure and record photoplethysmographic (PPG) properties, electrocardiogram (ECG) properties, skin temperature (T) properties, acceleration/motion (ACC) properties and electrodermal activity (EDA) properties of the training object. The data acquisition step provides measured body condition information containing raw PPG data, raw ECG data, raw T data, raw ACC data and raw EDA data. In the embodiment, raw PPG data is a time series of PPG information recorded at a sampling rate of 50-100 Hz, ECG information is recorded at a sample rate of 100-500 Hz, raw EDA data is a time series of EDA information recorded at a sampling rate of 4-30Hz, raw T data is a time series of temperature information recorded at a sampling rate of 0.2-1 Hz and raw ACC data is a time series of acceleration information recorded at a sampling rate of 25-100 Hz. At the same time, it is recorded, whether the measured body condition information is associated with a perimenopausal state or with a symptom of a perimenopausal state, either deriving from the affiliation of the training object to one of the two groups of experiencing/not experiencing a perimenopausal state or indication that the training object currently experiences/does not experience perimenopausal symptoms.

In a data preprocessing step S104, the measured body condition information is preprocessed to gain preprocessed body condition information. The data preprocessing step S104 is described in more detail by referring to FIG. 4.

In a filtering sub step S1041, the measured body condition information is subjected to a low pass filter to reduce signal noise. In the embodiment, the low pass filter is a Butterworth low pass filter with a cut off frequency of 0.5 Hz.

In an upgrading sub step S1042, the measured or hitherto preprocessed body condition information is aggregated to higher level information. The raw PPG data is converted to a time series of heart rate information, heart rate variability and blood pressure (with time). The raw T data is converted to a time series of body temperature information. The raw ACC data is converted to a time series of activity information, i.e. information that indicate a level of body activity over time. The raw EDA data is converted to time series of electrodermal phasic sympathetic activity (SCR) information and electrodermal tonic sympathetic activity (SCL) information.

In a sample reduction sub step S1043, the sampling rate of the measured or hitherto preprocessed body condition information is synchronized and reduced to 1 Hz. This considerably reduces the amount of data intended to be processed.

In a windowing sub step S1044, the time series of hitherto preprocessed body condition information is smoothened by determining a simple moving average (SMA) by forming the unweighted mean of the previous 60 data points, i.e., the unweighted mean of the values of preceding 60 samples of the measurements of each parameter.

In a labelling sub step S1045, the measured or hitherto preprocessed body condition information of 180 seconds respectively preceding or succeeding the detection of occurrence of a perimenopausal symptoms are labelled as belonging to a perimenopausal symptom as classification information. I.e. the point in time at that the training object detects and indicates occurrence of a perimenopausal symptom is recorded. Those measured or hitherto preprocessed body condition information, i.e. any sample thereof occurring in a time span of 180 seconds respectively preceding or succeeding that point in time is marked as belonging to a perimenopausal symptom, e.g. by setting a labelling bit to 1, while samples of measured or preprocessed body condition information outside of this 360 second time period are marked as not belonging to a perimenopausal symptom, e.g. by setting the labelling bit to 0. Thus, samples of measured or hitherto preprocessed body condition information are provided with classification information indicating whether the respective sample belongs to a symptom or not.

In an optional filtering sub step S1046, additional filtering of the measured or hitherto preprocessed body condition information or preprocessed body condition information may be applied in accordance with the measured body condition information or preprocessed body condition information. Thus, a peak in the time series of body temperature information may be dampened if it occurs together with a peak in the time series of activity information, in order to eliminate body temperature variations that follow from physical activity.

Additionally, or alternatively, a peak in the time series of body temperature information may be dampened if it occurs together with a rise of the ambient temperature if corresponding information is available, in order to eliminate body temperature variations that follow from ambient temperature variations.

Additionally, or alternatively, a peak in the time series of SCR information or SCL information may be dampened that occurs together with a rise in ambient temperature or a peak in activity information, in order to eliminate EDA variations that follow from increased perspiration following from physical activity or increased ambient temperature.

By the preprocessing step S104, the amount of data intended to be processed is significantly reduced in comparison to the related art approaches. Nevertheless, due to the specific data preprocessing, reliable classification results are still achieved. In particular in conjunction with the sensor unit 2 and the sensor location 52, reliable classification results are still achieved.

In a training step S105, a computer implemented classification model is generated. The training step S104 is described in more detail by referring to FIG. 5.

In a preparation sub step S1051, a computer implemented self-learning classification model is established. In the current embodiment, the self-learning classification model is a random forest model. In this random forest model, a set of decision trees is modelled such that a subset of features of the available preprocessed information is selected at random and assigned to the tree nodes.

In a feature input step S1052, the preprocessed body condition information consisting of a sample of a set of preprocessed body condition information of labelling sub step S1045 is input to the random forest model as training input information.

In a classification input step S1053, the corresponding classification information assigned to the training input information is input to the random forest model as training classification information. I.e., it is input whether the set of preprocessed body condition information of feature input step S1052 belongs to measured body condition information that was measured during occurrence of a menopausal state.

In a model adaption step S1054, the random forest model is adapted to the training input information and training classification information.

Feature input step S1052, classification input step S1053 and model adaption step S1054 are performed for all preprocessed body condition information.

As a result, there is a trained classification model to classify a perimenopausal state or the absence thereof. The thus gained classification model is used to diagnose a perimenopausal state of diagnose objects.

By means of FIG. 6, an embodiment of a computer implemented method for determining a menopausal state of a diagnose object is described.

In a classification model provision step S201, a classification model is trained according to the previously described method according to the method steps S101 through S105.

In a sensor positioning step S202, a sensor unit 2 is positioned at the sensor location 52 at a body of the diagnose object and is activated. The sensor location is positioned at the torso, particularly at the lower thorax, particularly below the sternum, either centrally or at the frontal left side of the thorax.

In a diagnose data acquisition step S203, the set of measurable body conditions are measured and recorded, i.e., photoplethysmographic (PPG) properties, electrocardiogram (ECG) properties, skin temperature (T) properties, acceleration/motion (ACC) properties and electrodermal activity (EDA) properties of the diagnose object. The diagnose data acquisition step provides measured diagnose body condition information containing raw PPG data, raw ECG data, raw T data, raw ACC data and raw EDA data. In the embodiment, raw PPG data is a time series of PPG information recorded at a sampling rate of 50-100 Hz, raw ECG information is is a time series of recorded at a sample rate of 100-500 Hz, raw EDA data is a time series of EDA information recorded at a sampling rate of 4-30 Hz, raw T data is a time series of temperature information recorded at a sampling rate of 0.2-1 Hz and raw ACC data is a time series of acceleration information recorded at a sampling rate of 25-100 Hz.

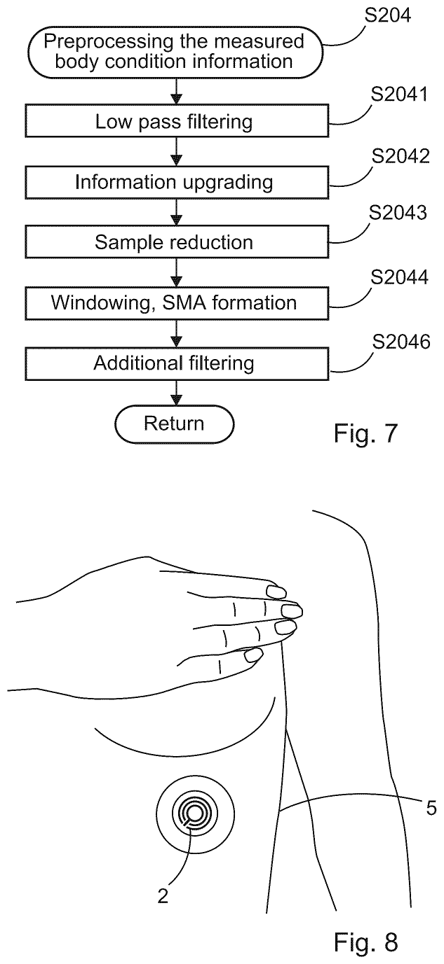

In a diagnose data preprocessing step S204, the measured body condition information is preprocessed to gain preprocessed diagnose body condition information. The diagnose data preprocessing step S204 is similar to the data preprocessing step S104 as described above by referring to FIG. 4 with some deviations as described by referring to FIG. 7.

In a filtering sub step S2041, the measured body condition information is subjected to a low pass filter to reduce signal noise. In the embodiment, the low pass filter is a Butterworth low pass filter with a cut off frequency of 0.5 Hz.

In an upgrading sub step S2042, the measured or hitherto preprocessed body condition information is aggregated to higher level information. The raw PPG data is converted to a time series of heart rate information, heart rate variability and blood pressure (with time). The raw T data is converted to a time series of body temperature information. The raw ACC data is converted to a time series of activity information, i.e. information that indicate a level of body activity over time. The raw EDA data is converted to time series of electrodermal phasic sympathetic activity (SCR) information and electrodermal tonic sympathetic activity (SCL) information.

In a sample reduction sub step S2043, the sampling rate of the measured or hitherto preprocessed body condition information is synchronized and reduced to 1 Hz. This considerably reduces the amount of data intended to be processed.

In a windowing sub step S2044, the time series of hitherto preprocessed body condition information is smoothened by determining a simple moving average (SMA) by forming the unweighted mean of the previous 60 data points, i.e., the unweighted mean of the values of preceding 60 samples of the measurements of each parameter.

In an optional filtering sub step S1046, additional filtering of the measured or hitherto preprocessed body condition information or preprocessed body condition information may be applied in accordance with the measured body condition information or preprocessed body condition information. Thus, a peak in the time series of body temperature information may be dampened if it occurs together with a peak in the time series of activity information, in order to eliminate body temperature variations that follow from physical activity.

Additionally, or alternatively, a peak in the time series of body temperature information may be dampened if it occurs together with a rise of the ambient temperature if corresponding information is available, in order to eliminate body temperature variations that follow from ambient temperature variations.

Additionally, or alternatively, a peak in the time series of SCR information or SCL information may be dampened that occurs together with a rise in ambient temperature or a peak in activity information, in order to eliminate EDA variations that follow from increased perspiration following from physical activity or increased ambient temperature.

By the preprocessing step S104, the amount of data intended to be processed is significantly reduced in comparison to the related art approaches. Nevertheless, due to the specific data preprocessing, reliable classification results are still achieved. In particular in conjunction with the sensor unit 2 and the sensor location 52, reliable classification results are still achieved.

In a diagnose data classifying step S205, samples of sets of the preprocessed diagnose body condition information are input into the classification model and the perimenopausal state of the diagnose object is classified by the classification model as diagnose classification information. For that, in the embodiment, the preprocessed diagnose body condition information is input into the random forest model and each decision tree of the random forest model is processed. Each decision tree achieves a local classification classifying the diagnose object either being in a perimenopausal state or not being in a perimenopausal state. The classification result of the random forest model is determined to be the one classification that is adopted by the majority of the decision trees.

The diagnose classification is recorded, output to the test object, e.g., via the transmission unit 3 or to an attending physician.

It occurs that in contrast to comparable fields like anxiety detection, where electrodermal phasic sympathetic activity is a significant marker, in detection of a perimenopausal state, surprisingly electrodermal tonic sympathetic activity is the more significant marker.

The invention was described by means of an embodiment. The embodiment is only of explanatory nature and does not restrict the invention as defined by the claims. As recognizable by the skilled person, deviations from the embodiment are possible without leaving the invention that is defined according to the scope of the claimed object-matter.

For example, in the embodiment, a random forest model was used as a classification model. Random forest models provide reliable classification results with moderate computational resource requirements. Alternatively, a decision tree may be employed as a classification model, requiring less computational resources. Alternative, deep learning methods and an artificial neuronal network may be employed as a classification model, providing an improved scalability.

In the embodiment, raw sensor data recorded by the sensor unit 2 is transmitted to the evaluation unit 4 via the transmission unit 3 and is pre-processed at the evaluation unit 4. Alternatively, the raw sensor data may be pre-processed by the sensor unit 2 or the transmission unit 3 to reduce the required data traffic.

In the embodiment, the menopausal state of perimenopause was classified. Alternatively, with corresponding training data, premenopause or postmenopause may be classified.

In this document, the terms “and”, “or” and “either . . . or” are used as conjunctions in a meaning similar to the logical conjunctions “AND”, “OR” (often also “and/or”) or “XOR”, respectively. In particular, in contrast to “either . . . or”, the term “or” also includes occurrence of both operands (“and/or”).

Method steps indicated in the description or the claims only serve an enumerative purpose of the method steps. They only imply a given sequence or an order where their sequence or order is explicitly expressed or is—obvious for the skilled person—mandatory due to their nature. In particular, the listing of method steps do not imply that this listing is exhaustive. Further steps may be interposed. Also, not all method steps described in an embodiment are required to implement the invention. The required method steps are defined by the claims.

In the description and claims, the term “comprising” does not exclude existence or presence of other elements or steps. The indefinite article “a” or “an” does not exclude a plurality and has to be understood as “at least one”.

Further, in this document, the following terms are understood in the following given meaning.

Menopausal state is the condition of a human body concerning the age-related loss of capability of ovarian generation of fertile ova through ovulation, usually recognized by the last occurrence of a menstruation. In the scope of this document, three menopausal states are distinguished: premenopause, perimenopause and postmenopause. Different menopausal states may exhibit different symptoms, respectively, and provide different options for mitigation of possible disorders, respectively.

Premenopause denotes a time period consisting of years leading up to the last menstruation, when the levels of reproductive hormones are becoming more variable and lower, and the effects of hormone withdrawal are present. Premenopause starts some time before the menstrual cycles become noticeably irregular in timing.

Perimenopause denotes a time period of the menopausal transition years before the date of the final menstruation. This transition may, dependent on the source, last for about four to eight years or six-to ten-years, ending 12 months after the last menstruation.

Postmenopause denotes a time period starting 12 months after the final menstruation, also indicated by a considerably increased FSH level of 16.74-113.59 mlU/ml.

Measurable body condition is a property of a human body that is accessible to a measurement by a sensor. Examples for measurable body conditions are skin temperature measurable by a thermometer, skin conductance/electrodermal activity like EDR and EDL, skin reflectance/skin light absorbance measurable by PPG, electric current flow through the heart measurable by ECG, tissue layer thicknesses measurable by ultrasonic sensors, liquid flow rates like blood flow rates measurable by ultrasonic sensors, acoustic noise emission spectrum as measurable by acoustic sensors or stethoscopes, body movement activity measurable by accelerometers attached to a human body, or the like.

Measured body condition information is data that a sensor directly or indirectly derives and outputs when measuring a measurable body condition. This data may be directly measured information, e.g. a value of light reflectance/absorbance of human skin, or deduced information like blood volume changes or blood oxygen saturation derived from changes of light reflectance/absorbance of human skin.

Skin conductance (SC)/electrodermal activity (EDA) is the property of the human body that causes continuous variation in the electrical characteristics of the skin. EDA may be measured electrically, alternatively or cumulatively by skin potential, skin resistance, skin conductance, skin admittance and skin impedance.

Electrodermal phasic sympathetic activity (EDR, SCR) are short lasting changes in EDA giving an objective, transient indication of autonomic nervous system arousal in response to a stimulus.

Electrodermal tonic sympathetic activity (EDL, SCL) are long lasting changes in EDA. They are generally considered to be the level of skin conductance in the absence of any particular discrete environmental event or external stimuli.

Photoplethysmography (PPG) is the optically obtaining of a plethysmogram that can be used to detect blood volume changes in the microvascular bed of tissue by measuring the property of the human body's skin to absorb/reflect light.

Electrocardiogram (ECG) is a measure of the electrical current that spreads through the heart during a cardiac impulse. A small amount of this current spreads to the skin and the electrical potential generated by the current can be detected by electrodes placed on the skin at opposite sides of the heart.

Classification describes the allocation of an object, e.g., an examined human, to a group of objects having one or more predetermined properties, e.g. to humans having a specific menopausal state.

Decision tree models are a known supervised learning approach used in statistics, data mining and machine learning. In this formalism, a classification or regression decision tree is used as a predictive model to draw conclusions about a set of observations. Tree models where the target variable can take a discrete set of values are called classification trees; in these tree structures, leaves represent class labels and branches represent conjunctions of features that lead to those class labels. Decision trees where the target variable can take continuous values (typically real numbers) are called regression trees. More generally, the concept of regression tree can be extended to any kind of object equipped with pairwise dissimilarities such as categorical sequences.

Random forest models are a known ensemble learning method for classification, regression and other tasks that operate by constructing a multitude of decision trees at training time. For classification tasks, the output of the random forest is the class selected by most trees. For regression tasks, the mean or average prediction of the individual trees is returned.

Deep learning models are a known family of machine learning methods based on artificial neural networks with representation learning. Learning can be supervised, semi-supervised or unsupervised. Deep learning models are a class of machine learning algorithms that use multiple layers to progressively extract higher-level features from a raw input.

### LIST OF REFERENCE SIGNS

1 computer implemented system for determining a menopausal state (first embodiment)
2 sensor unit
21 adhesive layer
22 sensing portion
23 sensor stored energy source, battery
24 sensor communication portion
3 transmission unit, mobile device, smart phone
31 short range communication portion
32 long range communication portion, Wi-Fi module, cellphone module
33 cellphone energy source, battery
4 evaluation unit
41 network interface
42 data storage
43 classification model
5 human body
51 dermal surface
52 sensor location
6 short range transmission link
7 long range transmission link, GSM network, internet

1. A computer implemented method for providing a model for determining a menopausal state, comprising the steps:
a) determining a set of measurable body conditions from a group of body conditions of a human body, the body conditions being indicative of a menopausal state of a human body,
b) determining a set of training objects, the training objects being humans being capable of adopting a menopausal state and having a known state concerning their menopausal state as menopausal state information,
c) measuring the measurable body conditions of the set of measurable body conditions of each of the training objects for a predetermined amount of time to provide measured body condition information for each of the training objects, and detecting occurrence of a symptom of a menopausal state,
d) preprocessing the measured body condition information to provide preprocessed body condition information,
e) providing a computer implemented classification model adapted to classify a menopausal state,
f) inputting the preprocessed body condition information of a training object as training input information to the classification model,
g) inputting the detected occurrence of the symptom of a menopausal state as training classification information to the classification model,
h) adapting the classification model according to the training input information and training classification information.

2. The method according to claim 1, wherein
in step c), the measured body condition information is a time series of measured body conditions, respectively.

3. The method according to claim 1, wherein
in step d) the computer implemented classification model is a decision tree model or a random forest model or an artificial neural network model, in particular a recurrent neural network model or a convolutional neural network model, further in particular an echo state network model or a liquid state machine model.

4. The method according to claim 1, wherein
in step a), the set of body condition information contains a subset of the set:
electrodermal activity,
heart rate, HRV and Blood Pressure, in particular photoplethysmographic information and ECG information
body temperature,
body movement activity, in particular relocation or acceleration, or
body environment information, in particular ambient temperature, ambient humidity or ambient pressure.

5. The method according to claim 1,
wherein step c) further comprises the step:
ca) measuring electrodermal activity and providing measured electrodermal activity information,
and step d) further comprises the steps:
da) transforming the measured electrodermal activity information into SCL information and SCR information, and
db) providing the SCL information or the SCR Information as measured body condition information,
and step f) comprises the step:
fa) inputting the SCL information or the SCR information as measured body condition information to the classification model.

6. The method according to claim 1,
wherein step h) further comprises the step:
ha) providing the SCL information with the highest weight of the set of measurable body conditions for classification.

7. The method according to claim 1,
wherein step d) further comprises at least one of the steps:
dc) applying a low pass filter, in particular a Butterworth filter, in particular a Butterworth filter with a cut off frequency of 0.5 Hz, to the measured body condition information,
dd) adapting a sampling rate of the measured body condition information to 1 Hz for the measurable body conditions of the set of measurable body conditions,
de) smoothen the measured body condition information by applying a sliding left windows of 60 seconds, in particular by determining a simple moving average by forming the unweighted mean of the previous 60 measured samples,
df) providing the measured body condition information with classification information such that the measured body condition information that occur within a time period of 180 seconds preceding and of 180 seconds succeeding a point in time of the detection of a symptom of a menopausal state are labeled as belonging to the symptom of the menopausal state, and that the measured body condition information that occur outside that time period are labeled as not belonging to a symptom of a menopausal state.

8. A computer implemented system for determining a menopausal state, comprising:
a sensor unit,
a transmission unit, and
an evaluation unit, containing a classification model provided according to claim 1,
wherein the system is configured to perform the steps:
i) determining a diagnose object, the diagnose object being a human being capable of adopting a menopausal state and having an unknown state concerning their menopausal state as menopausal state information,
j) arranging at least one sensor at the body of the diagnose object at a sensor body location,
k) measuring, by means of the at least one sensor, a set of measurable body conditions from a group of body conditions of a human body, the body conditions being indicative of a menopausal state of a human body and being a subset of the set of measurable body conditions according to step a), as measured diagnose body condition information,
l) preprocessing the measured diagnose body condition information to provide preprocessed diagnose body condition information,
m) inputting the preprocessed diagnose body condition information of the diagnose object as diagnose input information to the classification model,
n) classifying a menopausal state of the diagnose object as menopausal state information,
o) using the classified menopausal state information for providing a diagnose on the menopausal state of the diagnose object, wherein
the sensor unit is configured to measure a set of measurable body conditions from a group of body conditions of a human body, the body conditions being indicative of a menopausal state of a human body and being a subset of the set of measurable body conditions and transmit measured body conditions as measured body condition information to the transmission unit,
the transmission unit is configured to receive the body condition information from the sensor unit and to transmit the body condition information to the evaluation unit, and
the sensor unit is configured to perform the step l) and the evaluation unit is configured to perform the steps n) and o).

9. The system according to claim 8, wherein in step l), the set of body condition information contains a subset of the set:
electrodermal activity,
heart rate, heart rate variability and blood pressure in particular photoplethysmographic information and ECG information
body temperature,
body movement activity, in particular relocation or acceleration, or
body environment information, in particular ambient temperature, ambient humidity or ambient pressure.

10. The system according to claim 8, wherein step l) further comprises the step:
la) measuring electrodermal activity and providing measured diagnose electrodermal activity information,
and step m) further comprises the steps:
ma) transforming the measured diagnose electrodermal activity information into SCL information and SCR information, and
mb) providing the SCL information or the SCR Information as measured diagnose body condition information,
and step n) comprises the step:
na) inputting the SCL information or the SCR information as measured diagnose body condition information to the classification model,
or step o) further comprises the step:
oa) considering the SCL information with the highest weight of the set of measurable body conditions for classification.

11. The system according to claim 8,
wherein step m) further comprises at least one of the steps:
mc) applying a low pass filter, in particular, a Butterworth filter, in particular a Butterworth filter with a cut-off frequency of 0.5 Hz, to the measured diagnose body condition information,
md) adapting a sampling rate of the measured diagnose body condition information to 1 Hz for the measurable body conditions of the set of measurable body conditions,
me) smoothen the measured diagnose body condition information by applying a sliding left window of 60 seconds, in particular by determining a simple moving average by forming the unweighted mean of the previous 60 measured samples,
mf) applying an additional filter to the measured diagnose body condition information based on measured diagnose body condition information, or
mg) providing a sensor unit at a sensor location, wherein the sensor location is arranged at a human body, in particular at a torso, in particular at the lower thorax, in particular below the sternum, either centrally or at the frontal left side of the thorax of the diagnose object.

12. The system according to claim 8, wherein the sensor unit, the transmission unit or the evaluation unit is configured to perform step m).

13. The system according to claim 8, wherein the sensor unit further comprises
a sensing portion at a proximal side of the sensor unit, the sensing portion comprising a convex shape, and
an urging portion that is configured to urge the sensing portion towards a dermal surface at a sensor location, such that the sensing portion indents the dermal surface.
